(19) United States
(12) Patent Application Publication
(10) Pub. No.: US 2005/0021109 A1
(43) Pub. Date: Jan. 27, 2005

(54) Implant according to the "thin client" principle

(76) Inventor: Jorgen Samsioe, Munchen (DE)

Correspondence Address: SCHIFF HARDIN LLP;Patent Department, 6600 Sears Tower, 233 South Wacker Drive, Chicago, IL, 60606, US

(21) Appl. No.: 10/895,525

(22) Filed: Jul. 21, 2004

(30) Foreign Application Priority Data
Jul. 21, 2003 (DE) 103 33 133.6

(51) Int. Cl. A61N 1/08

(52) U.S. Cl. 607/60

(57) ABSTRACT

An implant, particularly in the form of an implantable chip, for use in an animal or human body, comprises an input device with measurement probes to acquire at least one body variable, an externally controlled output stage in order to act on the body, and a transmission and reception stage for communication with a doctor or an external computer.

### BACKGROUND OF THE INVENTION

The invention concerns an implant, in particular in the form of an implantable chip, for use in animal or human bodies, as well as an appertaining method.

At present, implants are very expensive since they must function perfectly over a long service life. Moreover, they are rather static in function since they can often not react to changes in the environment or changed requirements of the patient.

### SUMMARY OF THE INVENTION

The invention is therefore based on the object to provide an implant that can be used with a relatively simple design in a most multifaceted manner, and utilizes a relatively small and cheap power unit that is implanted. An appertaining method is also provided.

To achieve this object, such an implant comprises an input device with measurement probes to acquire at least one body variable, an output stage in order to act on the body, and a transmission and reception stage for communication with a doctor or other medical personnel, or an external computer.

Via the inventive design, whereby the implant is based on the “thin client” principle (i.e., expressed simply, the device constitutes a dumb system to be directed by external commands), it is not necessary to realize control devices conceptualized as high-quality, expensive and failsafe in the chip to be implanted since the control ensues externally via a closed transmission-reception loop. The doctor or a clinical mainframe can naturally react very simply to any changes and effect modifications, both in the detection of the body variables and, in particular, in the area of the output stage.

In an embodiment of the invention, the output stage is provided with a dosage device and a medicine storage in order to effect a dosed medicine administration based on the body variables detected in the input device (thus, for example any blood values, electrical voltages, concentrations of specific chemical substances or the like). However, this medicine administration is not deliberately controlled by a computer device in the implant, but rather externally via the doctor or an external mainframe. However, this output stage is not limited to the pure medicine administration; rather, other actions could also occur via the output stage, such as special electrical excitation signals or the like.

A particular simplification according to an embodiment of the inventive implant results when the transmission and reception stage, as can be provided according to a further feature of the present invention, is fashioned two-stage, with an implanted first stage that communicates with an external detection device on the body, this detection device establishing a remote connection to the doctor or computer.

The detection device can very simply be a mobile telephone connected with the implant via Bluetooth or the like, this telephone in turn being capable of connecting with its normal telephone part with the doctor or computer. This two-stage design naturally simplifies the design and particularly the size of the transmission and reception stage of the implant, since this must only be in the position to directly bridge a few centimeters in the body to the detection device externally mounted on the body. In connection with the very simple (due to the “thin client” principle) input device and output stage, an inventive, versatilely usable and externally controllable implant can be realized on a small scale and also very cheaply.

In addition to the provision of a power supply that is integrated into the implant, an energy supply stage, via the transmission and reception device, could also be supplied with energy in a simpler manner, meaning this power supply stage obtains its supply energy from a part of the energy of the radio connection.

### DESCRIPTION OF THE DRAWINGS

Further advantages, features and details of the invention result from the subsequent specification of an exemplary embodiment as well as using the drawings.

FIG. 1 is a pictorial schematic view of an embodiment of the inventive principle, with an implant according to the “thin client” principle; and

FIG. 2 is a schematic block diagram illustrating the design of the implant.

### DETAILED DESCRIPTION OF THE PREFERRED EMBODIMENTS

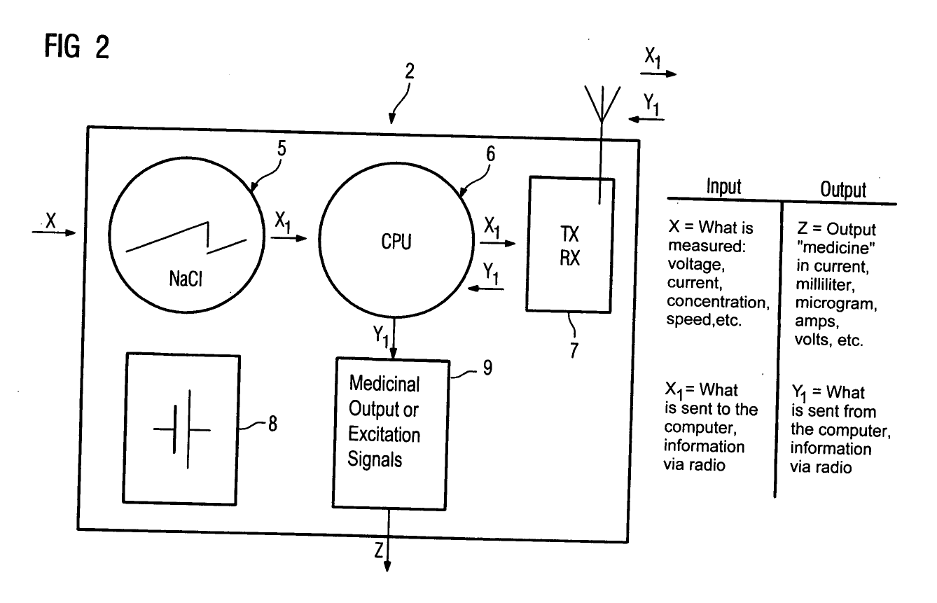

An implant 2 according to FIG. 2 is implanted in the patient 1 at a suitable location (for example, in an organ, a region of the heart, in a blood vessel or the like) that is connected via Bluetooth or the like with an external detection device 3 that can be attached directly to the body, for example, a mobile telephone. This detection device in turn maintains a remote connection to an external computer 4 that can, for example, be the mainframe of a clinic.

FIG. 2 schematically shows the design of an embodiment of the inventive implant 2, fashioned in the form of an implantable chip, with an input device 5 that, for example, can be provided with measurement probes to acquire at least one body variable X (as illustrated, a probe to measure the NaCl content in the blood), in a form such as voltage, current, concentration, speed, etc. An output stage 9 is provided that, for example, can comprise a medicine output but can also administer special excitation signals or the like to the body Z.

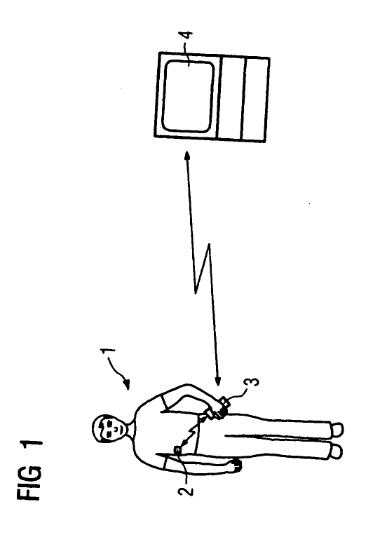

As a third significant unit, the implant comprises a transmission and reception stage 7 that is connected with a doctor or an external computer, whereby (as in FIG. 1) this connection is preferably fashioned in a two-stage manner, with a near connection stage to a detection device 3 and a remote connection stage to an actual computer 4. The transmission and reception stage 7 is configured to communicate information X1 wirelessly and receive information Y1 wirelessly.

The input device 5, the output device 9 and the transmission and reception stage 7 are connected with one another via a CPU (central processing unit) 6.

According to this embodiment, it is significant that the control of the output stage 9 based on the body variables determined by the input device 5 does not ensue within the chip 2, but rather that the input values X are sent out X1 by the transmission and reception stage 7, calculated there, and finally the corresponding commands are externally acquired again Y1 by the transmission and reception stage 7 and used to control the output stage 9. As necessary, a switching of the input device 5 with its measurement probes to detect at least one body variable can also externally ensue.

An integrated power source 8 (for example, a battery) may be provided.

For the purposes of promoting an understanding of the principles of the invention, reference has been made to the preferred embodiments illustrated in the drawings, and specific language has been used to describe these embodiments. However, no limitation of the scope of the invention is intended by this specific language, and the invention should be construed to encompass all embodiments that would normally occur to one of ordinary skill in the art.

The present invention may be described in terms of functional block components and various processing steps. Such functional blocks may be realized by any number of hardware and/or software components configured to perform the specified functions. For example, the present invention may employ various integrated circuit components, e.g., memory elements, processing elements, logic elements, look-up tables, and the like, which may carry out a variety of functions under the control of one or more microprocessors or other control devices. Similarly, where the elements of the present invention are implemented using software programming or software elements the invention may be implemented with any programming or scripting language such as C, C++, Java, assembler, or the like, with the various algorithms being implemented with any combination of data structures, objects, processes, routines or other programming elements. Furthermore, the present invention could employ any number of conventional techniques for electronics configuration, signal processing and/or control, data processing and the like.

The particular implementations shown and described herein are illustrative examples of the invention and are not intended to otherwise limit the scope of the invention in any way. For the sake of brevity, conventional electronics, control systems, software development and other functional aspects of the systems (and components of the individual operating components of the systems) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the invention unless the element is specifically described as “essential” or “critical”. Numerous modifications and adaptations will be readily apparent to those skilled in this art without departing from the spirit and scope of the present invention.

### Reference List

1 patient
2 implant
3 detection device
4 external computer
5 input device
6 CPU
7 transmission and reception stage
8 power source
9 output stage

1. An implant formed as an implantable chip for use in an animal or human body, comprising:
an input device with measurement probes to acquire at least one body variable;
an externally controlled output stage in order to act on the body; and
a transmission and reception stage for communication with a doctor, other medical personnel, or an external computer.

2. The implant according to claim 1, wherein the implant is based on a “thin client” principle and is directed by external commands.

3. The implant according to claim 1, wherein the output stage comprises a dosing device and a medicine storage.

4. The implant according to claim 1, wherein the transmission and reception stage comprises two-stages: an implantable first stage configured to communicate with a detection device outside on the body, the detection device being a second stage configured for establishing a remote connection to the doctor, the other medical personnel, or the external computer.

5. The implant according to claim 4, wherein the detection device is a mobile telephone connected with the implant via Bluetooth.

6. A method for operating an implantable chip for use in an animal or human body, comprising:
acquiring at least one body variable with an input device;
communicating the at least one body variable to a doctor, other medical personnel, or an external computer with a transmission and reception stage;
receiving an external command from the doctor, the other medical personnel, or the external computer; and
acting on the body via an externally controlled output stage based on the external command.
